(19) United States
(12) Patent Application Publication
(10) Pub. No.: US 2012/0183624 A1
(43) Pub. Date: Jul. 19, 2012

(54) METHOD FOR MANUFACTURING HOMEOPATHIC PERSONAL CARE PRODUCTS

(75) Inventor: Peter Klapper, San Francisco, CA (US)

(73) Assignee: FORCES OF NATURE, INC., Sonoma, CA (US)

(21) Appl. No.: 13/429,282

(22) Filed: Mar. 23, 2012

(51) Int. Cl. A61K 36/14; A61K 35/56; A61K 36/185; A61K 33/14; A61K 36/28; A61K 36/77

(52) U.S. Cl. 424/520; 424/770; 424/725; 424/769; 424/680

(57) ABSTRACT

A method for making a personal care composition including homeopathic remedies and essential oils. The homeopathic remedies are selected from one or more pharmacopeias wherein the remedies are indicated for one or more biological conditions. Remedies are selected because they are indicated for the same disease condition or in certain embodiments related conditions. Once selected the remedies are diluted to a predetermined concentration and blended with essential oils. In certain embodiments a medicated cleansing bar, a soap, or a cleansing material may be selected for delivering the homeopathic remedy and the essential oils. The essential oils may be extracted from plant material using various techniques including distillation.

### BACKGROUND

The present invention relates generally to homeopathy and more particularly to a method of manufacturing and delivering homeopathic remedies through the use of personal care products.

Homeopathic medicines are well known, and conventionally manufactured using the Hahnemanian process. In general, the active homeopathic ingredient is dispersed in a carrier solution, generally, a solution of water and alcohol or an alkaloid mixture. Where the carrier solution is a water and alcohol base solution, the water is normally purified prior to mixing with the alcohol. The active homeopathic ingredient of the medicine is mixed with the carrier solution in the appropriate proportion to achieve the desired concentration of the active homeopathic ingredient in the carrier solution.

A 1× potency homeopathic medicine is a solution which comprises one part of active homeopathic ingredient to nine parts of carrier solution. A 2× potency homeopathic medicine is a solution which comprises one part of active homeopathic ingredient to ninety-nine parts of carrier solution. A 3× potency homeopathic solution is one which comprises one part active homeopathic ingredient to nine hundred and ninety-nine parts carrier solution. An N× potency homeopathic medicine is a solution of one part of active homeopathic ingredient to (10N-1) parts of carrier solution.

Essential oils are substances comprising a volatile odorant characteristic extracted from a plant. Interest in essential oils has revived in recent decades with the popularity of aromatherapy. Often the oils are volatilized or diluted in a carrier oil and used in massage, diffused in the air by a nebulizer, heated over a candle flame, or burned as incense.

Personal care products include soaps, cleansing bars, topical solutions, and mixtures for applying remedies to skin and other tissues.

### SUMMARY

Disclosed herein is a method for making personal care compositions including, but not limited to, homeopathic remedies and essential oils. The homeopathic remedies are selected from one or more pharmacopeias wherein the remedies are indicated for one or more biological conditions. Remedies are selected because they are indicated for the same disease condition or, in certain embodiments, related conditions. Once selected the remedies are diluted to a predetermined concentration and blended with essential oils. In certain embodiments a carrier may be blended into the topical preparation. The carrier may be a relatively low volatility oil extracted from fruit or seed material. The essential oils may be extracted from plant material using various techniques including but not limited to distillation.

In certain embodiments the method may include selecting a plurality of homeopathic remedies, each of said homeopathic remedies indicated for a particular biological condition, selecting a plurality of essential oils; and blending the remedies and oils to product a topical preparation.

In some embodiments the homeopathic remedy and essential oil may be added to soap, cleansing bars, or soap-like compositions to provide a means to apply the remedies topically.

The construction and method of operation of the invention, however, together with additional objectives and advantages thereof will be best understood from the following description of specific embodiments when read in connection with the accompanying drawings.

### BRIEF DESCRIPTION OF THE DRAWINGS

FIG. 1 shows steps in a process according to certain embodiments disclosed herein.

### DESCRIPTION

### Generality of Invention

This application should be read in the most general possible form. This includes, without limitation, the following:

References to specific techniques include alternative and more general techniques, especially when discussing aspects of the invention, or how the invention might be made or used.

References to “preferred” techniques generally mean that the inventor contemplates using those techniques, and thinks they are best for the intended application. This does not exclude other techniques for the invention, and does not mean that those techniques are necessarily essential or would be preferred in all circumstances.

References to contemplated causes and effects for some implementations do not preclude other causes or effects that might occur in other implementations.

References to reasons for using particular techniques do not preclude other reasons or techniques, even if completely contrary, where circumstances would indicate that the stated reasons or techniques are not as applicable.

Furthermore, the invention is in no way limited to the specifics of any particular embodiments and examples disclosed herein. Many other variations are possible which remain within the content, scope and spirit of the invention, and these variations would become clear to those skilled in the art after perusal of this application.

Specific examples of components and arrangements are described below to simplify the present disclosure. These are, of course, merely examples and are not intended to be limiting. In addition, the present disclosure may repeat reference numerals and/or letters in the various examples. This repetition is for the purpose of simplicity and clarity and does not in itself dictate a relationship between the various embodiments and/or configurations discussed.

Read this application with the following terms and phrases in their most general form. The general meaning of each of these terms or phrases is illustrative, not in any way limiting.

### Lexicography

The term “Extraction” generally means a separation process consisting of the separation of a substance from a matrix. Conventional extraction techniques include, but are not limited to liquid-liquid extraction, solid phase extraction, supercritical carbon dioxide extraction, ultrasonic extraction, heat reflux extraction, or microwave-assisted extraction.

The term “Topical” generally means designed for application to body surfaces such as the skin, organs or membranes. Topical medications may also be inhalational or applied to the surface of tissues. Topical is contrasted with enteral (in the digestive tract) and parenteral (injected into the circulatory system).

The terms “Homeopathic Drug” or “Homeopathic Remedy” generally refer to any drug labeled as being homeopathic and/or using homeopathic material listed in recognized homeopathic pharmacopeias such as the Homeopathic Pharmacopeia of the United States (HPUS), an addendum to it, or its supplements. The potencies of homeopathic drugs are specified in terms of dilution, i.e., 1× ( 1/10 dilution), 2× ( 1/100 dilution), etc.

The term “Homeotherapeutics” generally refers to therapies which utilizes drugs that are selected and administered in accordance with the tenets of homeopathy or as described in various pharmacopeias.

### DETAILED DESCRIPTION

### Essential Oils

An essential oil is generally a concentrated hydrophobic liquid containing volatile aroma compounds from plants. The process of oil extraction may involve steam distillation from leaves or other parts of a plant. Accordingly, essential oils may also be known as volatile oils or ethereal oils. The term “essential” is applied to convey the idea that the oil has distinctive scent, or essence, of a plant.

While various essential oils have been used medicinally at different periods in history, essential oils do not form a distinctive category for any medical or pharmacological purpose. Claims for the efficacy of medical treatments and treatment of cancers in particular, are now subject to regulation in most countries. As the use of essential oils has declined under the scrutiny of evidence-based medicine, current information providers are less likely to generalize the phrase “essential oils” and instead name the specific compound that characterizes the oil. For example, instead of calling the compound “Oil of Wintergreen”, the term methyl salicylate will be used.

Various extraction methods are used in the manufacture and extraction of essential oils. In certain embodiments essential oils are extracted by distillation. Other embodiments may include expression, or solvent extraction. Raw plant material, consisting of the flowers, leaves, wood, bark, roots, seeds, or peel, is put into a distillation apparatus (alembic) over water. By passing steam through the plant material, the volatile compounds are vaporized. The vapors flow through a condensation coil, where they condense back to liquid, which is then collected. The extraction method may alter the characteristics of the oil and the strength (potency) of the essence of the extract. In certain embodiments the extraction of essential oils determines the quality of the oil that is used, since a poorly executed extraction can damage the oil and alter the chemical signature of the essential oil.

Extraction methods may include, without limitation, one or more of the following:
Distillation
Water distillation
Steam distillation
Hydro diffusion
Cohobation
Rectification
Fractional distillation
Sponge expression
Écuelle à piquer
Machine abrasion
Solvent extraction
Maceration
Enfleurage
Hypercritical carbon dioxide CO2

Accordingly certain embodiments according to the current disclosure may include oils from the same or similar plants extracted using multiple methodologies. For example and without limitation, an essential oil of wintergreen may be extracted using a distillation process and then combined with another oil of wintergreen extracted using solvent extraction.

References in the specification to “one embodiment”, “an embodiment”, “an example embodiment”, etc., indicate that the embodiment described may include a particular feature, structure or characteristic, but every embodiment may not necessarily include the particular feature, structure or characteristic. Moreover, such phrases are not necessarily referring to the same embodiment. Further, when a particular feature, structure or characteristic is described in connection with an embodiment, it is submitted that it is within the knowledge of one of ordinary skill in the art to effect such feature, structure or characteristic in connection with other embodiments whether or not explicitly described. Parts of the description are presented using terminology commonly employed by those of ordinary skill in the art to convey the substance of their work to others of ordinary skill in the art.

### Carrier Oil

Certain embodiments may use a carrier which may be another oil. Carrier oils are derived from the fruit or seeds of a plant. Conventionally, carrier oils do not contain a concentrated aroma, although some, such as olive oil, have a mild distinctive smell. Carrier oils are generally fixed oils, indicating a fatty oil of vegetable origin characterized by relatively low volatility. Carrier oils are generally extracted using cold-pressing. Carriers may be used to dilute an essential oil during production, packaging or application. Carrier oils may also be selected based on their evaporation rate because they do not evaporate at the same rate as an essential oil. Essential oils are relatively more volatile. In certain embodiments the carrier oil should be as natural and unadulterated as possible. Therefore certain embodiments employ all natural processes for growth and extraction of the oil.

The carrier selection process entails consideration of the area where the topical compound will be applied. In human use, sensitivity and viscosity may be dominant oil characteristics. For example and without limitation, grape seed oil is typically very thin, while olive oil is much thicker. Carrier oils may be easily blended to combine their properties of viscosity, acceptability, lubrication, absorption, aroma and so forth.

Carrier oils may include, without limitation, one or more of the following:
Sweet almond oil
Grape seed oil
Avocado oil
Olive oil
Sesame oil
Evening primrose
Canola (Rapeseed)
Sunflower oil
Jojoba oil
Castor oil
Walnut oil
Peanut oil
Pecan oil
Macadamia nut
Fractionated coconut oil
Hazel nut oil
Rose hip seed oil
Vegetable oil

Carrier oils are generally kept cool (below ambient), and away from strong light, to retard rancidification. In certain embodiments refrigerating oils helps preserve their freshness, however, very cold oils may appear cloudy and not be suitable for further processing.

### Homeopathy Remedies

Homeopathic remedies are regulated in the same manner as nonprescription, over-the-counter (OTC) drugs. In certain embodiments the active ingredients are found in the current Homeopathic Pharmacopeia of the United States (HPUS); however this list is not intended to be limiting in any way. Homeopathic remedies are prepared according to the guidelines in the Homeopathic Pharmacopeia of the United States (HPUS), which was written into law in the Federal Food, Drug, and Cosmetic Act in 1938. The U.S. Food and Drug Administration (FDA) requires that homeopathic remedies meet certain legal standards for strength, purity, and packaging. For example and without limitation, the labels on the remedies must include at least one major indication (i.e., medical problem or biological condition to be treated), a list of ingredients, the dilution, and safety instructions. More than one remedy may be indicated for a particular medical problem or biological condition.

Beside the Homeopathic Pharmacopeia of the United States, certain embodiments may include homeopathic remedies from other pharmacopoeias, including but not limited to:
The German Homeopathic Pharmacopeia (GHP)
The European Pharmacopeia
The French Homeopathic Pharmacopeia, or the
The British Homeopathic Pharmacopeia.

A base preparation of a homeopathic remedy may be made by liquid extraction in a solvent. The process may include extracting the ingredients in a suitable solvent, such as an alcohol, a water-alcohol mixture, water, glycerin, or isotonic sodium chloride solution. Once extracted, comminution, maceration, squeezing, or grinding may be employed to reach a desired concentration. In certain embodiments grinding with lactose may be performed with the effect of creating a powdered mixture.

Certain homeopathic remedies are commercially available. For example and without limitation Natrum Muriaticum, Thuja Occidentalis, and Calendula Officinalis are all commercially available as liquids, pellets or other solid form. Others may be in gel form. These solids may be solubilized or prepared as an emulsion by combining with an appropriate carrier. The nature of the homeopathic remedy may determine the appropriate carrier. For example and without limitation, alcohols are used for hydrophobic remedies and water for others.

Once the homeopathic remedy is prepared as a liquid, various dilutions may be performed to achieve a desired concentration. This may be effectuated with a series of dilutions prepared from the base preparation. Conventional homeopathic remedies use dilute solutions reducing concentrations to less than 1 ppm, however lower concentrations may also be effected. Trace amount (barely discernable) solutions may also be prepared. In certain embodiments the base preparation may be relatively concentrated including concentrations as high as 60% homeopathic remedy.

### Remedy Selection

Homeopathic remedies have generally recognized affects set forth in the HPUS among other pharmacopeias, however, those skilled in the art of homeopathy may recognize other affects not widely recognized. In any event, certain embodiments include combinations of more than a single homeopathic remedy that are targeted towards a particular biological condition. For example if Natrum Muriaticum, Thuja Occidentalis, and Calendula Officinalis are all used for treating certain skin conditions, then those homeopathic remedies may be combined. Similarly if Calendula Officinalis, Phytolacca Americana, and Thuja Occidentalis are each known for treating warts and promoting healthy skin (as a consequence of the warts), then certain embodiments may include low dosage amounts of those homeopathic remedies.

### Cleansing Material

A soap, cleansing bar or cleansing material is generally a salt of a fatty acid used as surfactants for washing and cleaning. Conventionally they are obtained by treating vegetable or animal oils and fats with a strongly alkaline solution. Because fats and oils are composed of triglycerides (three molecules of fatty acids attached to a single molecule of glycerol) the alkaline solution brings about saponification. In saponification, the fats are first hydrolyzed into free fatty acids, which then combine with the alkali to form crude soap. Glycerol, often called glycerine, is liberated and in certain embodiments may be left in or washed out of the resulting product.

When used for cleaning, the cleansing material serves as a surfactant (in conjunction with water) through the operation of micelles. Micelles are an aggregate of surfactant molecules dispersed in a liquid colloid. A typical micelle in aqueous solution forms an aggregate with the hydrophilic regions in contact with surrounding solvent, sequestering the hydrophobic regions in the micelle centre. The addition of soap allows oils to disperse in water and be rinsed away. Synthetic detergents operate by similar mechanisms to soaps or cleansing bars.

The type of alkali metal used in the manufacture of the cleansing material determines the kind of bar produced. Sodium soaps, which may be prepared from sodium hydroxide, are firm, whereas potassium soaps, which may be derived from potassium hydroxide, are softer or often liquid. Conventionally, potassium hydroxide was extracted from the ashes of bracken or other plants.

Cleansing materials are generally derivatives of fatty acids made from triglycerides (oils and fats). Triglyceride is often another name for the triesters of fatty acids and glycerin. For example, tallow, rendered beef fat, is the most available triglyceride from animals. Its saponified product is often called sodium tallowate. Conventional vegetable oils used in soap making are palm oil, coconut oil, olive oil, and laurel oil. Each species of oil offers quite different fatty acid content and, hence, results in cleansing bars of distinct feel. The seed oils give softer but milder bars. Bars made from pure olive oil are sometimes called Castile soap or Marseille soap and are reputed for being extra-mild. The term “Castile” is also sometimes applied to bars from a mixture of oils, but a high percentage of olive oil. Accordingly carrier oils may be used in the manufacture of bars in certain embodiments according to the current disclosure, however may not be required in other embodiments.

In certain embodiments homeopathic remedies may be added to a cleansing material. Solid or liquid cleansing material allows for topical application of homeopathic remedies directly to the applied surface or tissue. Moreover, the cleansing action may increase the effectiveness of the homeopathic remedy. In certain embodiments multiple homeopathic remedies, each indicated for the same or a related biological condition, may be added to the cleansing material. One having skill in the art will recognize that trace amounts or low concentrations of homeopathic remedy will meet the objectives of the current disclosure.

### Processing Steps

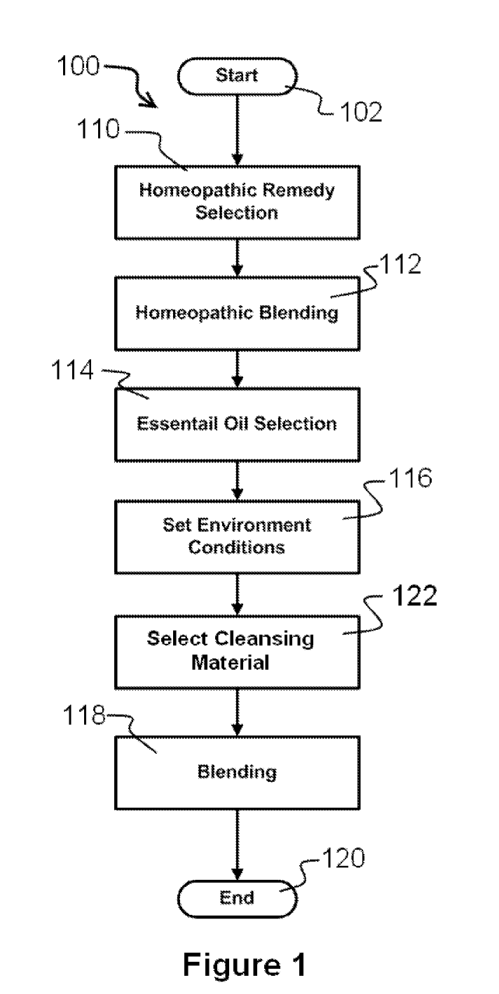

FIG. 1 shows steps in a process 100 according to certain embodiments disclosed herein. In FIG. 1 the method starts at a flow label 102. At a step 110 homeopathic remedy selections takes place. The selection process may be as described above wherein one or more homeopathic remedies, recognized for a particular affect, are selected to achieve relief of (or a change) in a biological condition. The particular affect of the homeopathic remedy may be as described in one or more recognized pharmacopeias such as the HPUS. In certain embodiments the homeopathic remedies might not target the same biological condition, but may be directed towards a related condition. For example and without limitation, an anti-wart remedy might be combined with a remedy for healing skin conditions such as inflammation or dryness.

At a step 112 the selected homeopathic remedies are blended. The blending step may include mixing if the homeopathic remedies are in liquid form. If the homeopathic remedies are in a solid or gel form then the blending step 112 may include the additional process of converting the remedy into a liquid form. Blending may be effectuated at room temperature in certain embodiments, while other may require an increased temperature to maintain suitable viscosity. One having skill in the art will recognize that in certain embodiments, it may be preferable to blend the homeopathic remedies in step 118.

At a step 114 essential oils are selected. The essential oils may be selected to complement or counteract scents from the homeopathic remedy, a disease condition, or a carrier oil (if used). Certain essential oils may mask or cover earthy, tart or sour smelling odors, while other essential oils provide sufficient fragrance to indicate to a user that the mixture has been applied topically.

At a step 116 environmental factors are established. Besides the proper temperature required for blending (see below) concentrations levels are established. In certain embodiments dilutions less than 1 ml of homeopathic remedy per liter of essential oil may be employed, however nothing in this disclosure should be read to limit the concentration or ratios of mixtures according to the current disclosure because different embodiments employ different concentrations. Moreover, the homeopathic remedy may be the result of the homeopathic blending of step 112 which may have the effect of providing a solution with different concentrations for each homeopathic remedy.

At a step 122 a cleansing material is selected. In certain embodiments, conventional or commercially available bars for treating certain biological conditions may be selected. For example and without limitation, acne cleansing bars such as those made from keratolytic or sulfur may be selected for use with homeopathic remedies indicated for acne. In other embodiments the cleansing material selection may relate to the biological condition the homeopathic remedy is designed to treat. For example and without limitation, a moisturizing bar may be employed for conditions requiring increased skin moisture. In yet other embodiments conventional soaps, portions of conventional soap recipes (saponified products), or cleansing bars may be used.

At a step 118 the essential oils and the homeopathic remedy is blended. At the step 118 carrier oils may be added. Blending may require an increase in temperature to effect a uniform result or to solubilize one or more of the ingredients. Blends performed at slightly above ambient may be employed in certain embodiments including a temperature range of 37 to 40 degrees Celsius. In addition, blending may require a commercial blending apparatus owing to the viscosity of the oils or carrier oil. Certain bars may require additional environmental conditions to effectuate proper blending.

At a flow marker 120 the method ends.

### Mixtures

In one mixture, effectuated using at least some of the steps in method 100 contains Thuja Occidentalis, Natrum Muriaticum, and Calendula Officinalis as the homeopathic remedies. Concentrations may range from 2× to 12× depending on the desired potency. They contain naturally occurring compounds such as thujone, camphene, d-sabinene, terpinen-4-ol and bornyl acetate. The essential oils may contain Cymbopogon Martini, Sri Lankan Cymbopogon Citratus, Lavandula Super, Pelargonium Graveolens, Melaleuca Alternifolia, and Pinus Sylvestris. The essential oils are rich in one or more of linalool, terpene oxides, terpene hydrocarbons, monoterpenoids, sesquiterpenes or phenolic compounds. A carrier oil may be included having the added affect of a more lipophilic formulation.

One having skill in the art will recognize that the concentrations given in the current disclosure may be varied to effect alternative embodiments of the mixture.

Yet another mixture may include Silicea 6× HPUS, Calendula Officinalis 6× as homeopathic remedies. The homeopathic ingredients may be blended with various concentrations of Cupressus Sempervirens, Rosmarinus Verbanone, Lavandula Super, Cymbopogon Martini, Melaleuca Alternifolia, Pelargonium Graveolens.

Yet another mixture made using at least some of the steps in method 100 includes the homeopathic remedies Hamamelis Virginiana which the HPUS indicates for relief of hemorrhoids, and Aesculus Hippocastanum also indicated for treatment of hemorrhoids. Those homeopathic remedies, among others, may be combined with the essential oils Cupressus Sempervirens, Lavandula Super, Pelargonium Graveolens, and Sesamum Indicum.

### Topical Delivery

The various embodiments describe herein may be delivered to a user topically where the resulting mixture is applied to the skin of a person to achieve the desired result. Certain embodiments include one or more essential oils creating a hydrophobic mixture. The combine mixture of homeopathic remedies combined with essential oils operates to maintain the homeopathic remedy on the skin thus increasing the time for effective operation.

In other embodiments the homeopathic remedy is delivered through the action of using the cleansing bar. These embodiments may combine a mixture of homeopathic remedies together with essential oils. In operation, the homeopathic remedies are not normally in contact with the target tissue because when the cleansing bar is rinsed off, the homeopathic remedy may be rinsed off as well.

The above illustration provides many different embodiments or embodiments for implementing different features of the invention. Specific embodiments of components and processes are described to help clarify the invention. These are, of course, merely embodiments and are not intended to limit the invention from that described in the claims.

Although the invention is illustrated and described herein as embodied in one or more specific examples, it is nevertheless not intended to be limited to the details shown, since various modifications and structural changes may be made therein without departing from the spirit of the invention and within the scope and range of equivalents of the claims. Accordingly, it is appropriate that the appended claims be construed broadly and in a manner consistent with the scope of the invention, as set forth in the following claims.

What is claimed is:

1. A method of preparing a personal care composition including:
selecting a plurality of homeopathic remedies, each of said homeopathic remedies indicated for a particular biological condition;
selecting a plurality of essential oils;
selecting a cleansing material, and
blending the remedies and oils into the cleansing material to produce a personal care composition.

2. The method of claim 1 wherein the remedies are in a substantially smaller concentration than the oils.

3. The method of claim 1 wherein the homeopathic remedy is in substantially trace amounts.

4. The method of claim 1 wherein the homeopathic remedy is between 1 PPM and 6 PPM of the personal care composition.

5. The method of claim 1 further including:
selecting a second homeopathic remedy indicated for a related condition, and
blending the second homeopathic remedy into the personal care composition.

6. The method of claim 5 wherein the cleansing material is a medicated cleansing material indicated for the same condition as one or more of the homeopathic remedies.

7. The method of claim 5 wherein the cleansing material is a medicated cleansing material indicated for a condition related to the one or more of the homeopathic remedies.

8. The method of claim 1 wherein the homeopathic remedy includes at least one of either hamamelis virginiana, aesculus hippocastanum, silicea, calendula officinalis, thuja occidentalis, or natrum muriaticum.

9. The method of claim 1 wherein the essential oil includes at least one of linalool, terpene hydrocarbons, monoterpenoids, sesquiterpenes, or phenolic compounds.

10. A method including:
preparing a homeopathic remedy, said homeopathic remedy indicated for treatment of a biological condition;
preparing one or more essential oils,
preparing a cleansing bar, and
mixing the homeopathic remedy and the one or more essential oils with the cleansing bar to create a personal care composition.

11. The method of claim 10 wherein the dilution of homeopathic remedy is in substantially trace amounts.

12. The method of claim 10 wherein the homeopathic remedy is between 1 PPM and 6 PPM of the personal care composition.

13. The method of claim 10 further including:
preparing a dilution of a second homeopathic remedy, said second homeopathic remedy indicated for treatment of a related biological condition, and
mixing the second homeopathic remedy in the personal care composition.

14. The method of claim 10 wherein the mixing is performed at a temperature between 20 and 25 degrees celsius.

15. The method of claim 10 wherein the mixing is performed at a temperature above 37 degrees celsius.

16. The method of claim 10 further including:
adding a carrier to the personal care composition.

17. The method of claim 16 wherein the carrier is an oil cold-press extracted from either a fruit or a seed.

18. The method of claim 10 wherein
the homeopathic remedies include at least one of either hamamelis virginiana, aesculus hippocastanum, silicea, calendula officinalis, thuja occidentalis, or natrum muriaticum, and
wherein the essential oil includes at least one of cupressus sempervirens, lavandula super, pelargonium graveolens, or sesamum indicum.
